(19) United States
(12) Plant Patent Application Publication
(10) Pub. No.: US 2006/0041982 P1
(43) Pub. Date: Feb. 23, 2006

(54) Kalanchoe plant named 'KJ 2003 0638'

(50) KALANCHOE BLOSSFELDIANA X LACINIATA; KALANCHOE

(75) Inventors: Knud Jepsen, Hinnerup (DK); Ellen Christensen, Hinnerup (DK)

Correspondence Address: FOLEY AND LARDNER LLP;SUITE 500, 3000 K STREET NW, WASHINGTON, DC, 20007, US

(73) Assignee: Knud Jepsen a/s

(21) Appl. No.: 11/011,611

(22) Filed: Dec. 15, 2004

(30) Foreign Application Priority Data
Aug. 18, 2004 (QZ) 2004/1546

(51) Int. Cl. A01H 5/00

(52) U.S. Cl. PLT/335

(57) ABSTRACT

A new and distinct K. blossfeldiana×K. laciniata interspecific hybrid plant named ‘KJ 2003 0638’ characterized by a large number of petals per flower resulting in the double-type or multi-petalled trait; large petal size resulting in a large flowers; the hastate and dissected young and mature leaves; the different soft-white and creamy colors of petals; and the large number of flowers on each plant.

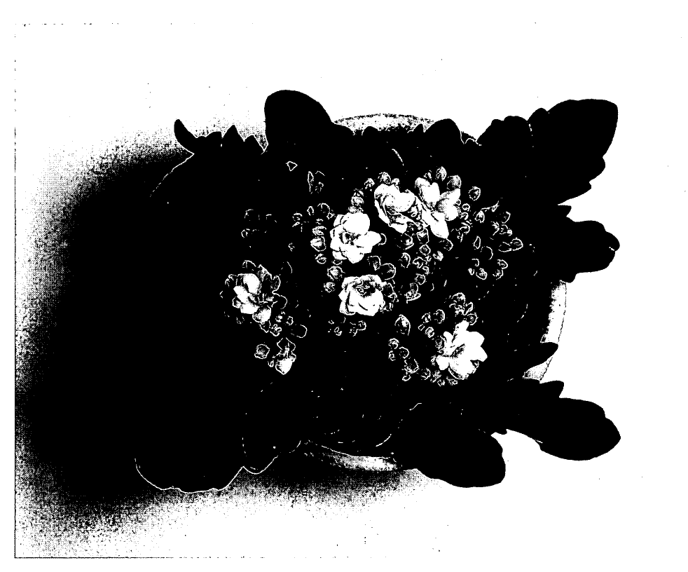

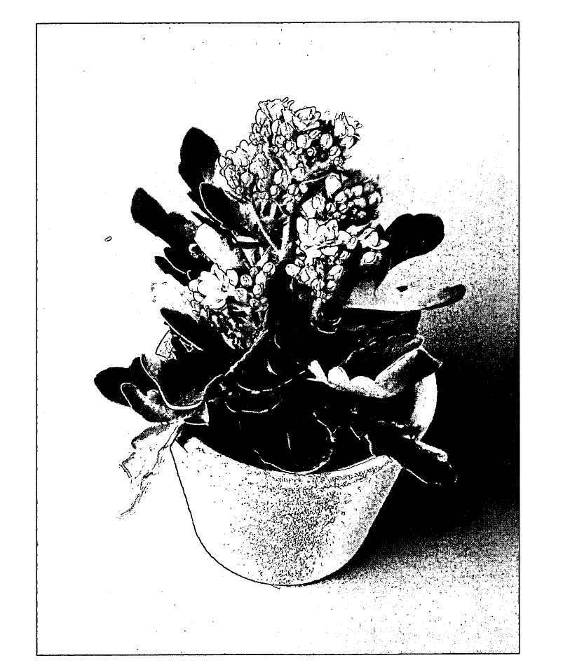

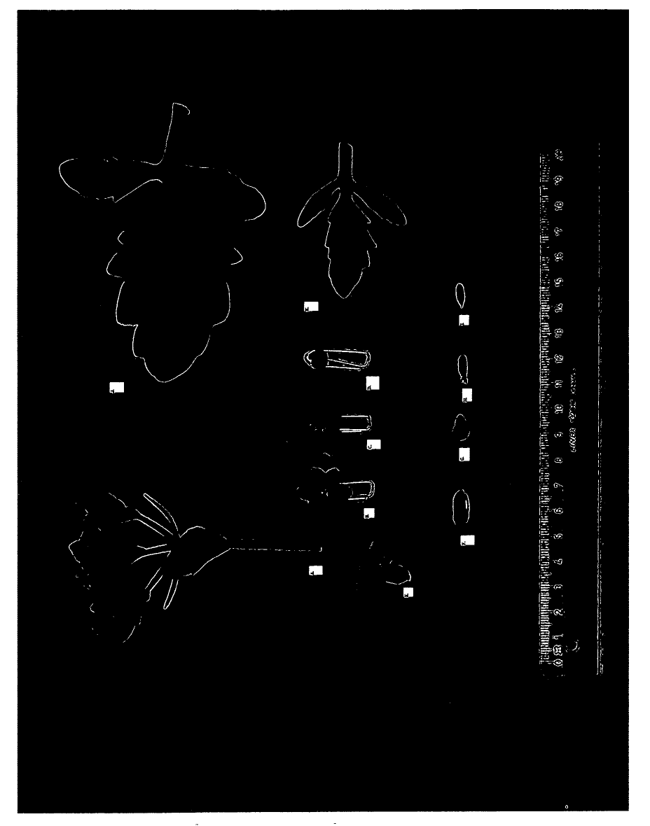

Latin name of the genus and species of the claimed plant:

K. blossfeldiana×K. laciniata interspecific hybrid.

Variety denomination:

‘KJ 2003 0638’

### BACKGROUND OF THE INVENTION

The present invention relates to a new and distinct cultivar of Kalanchoe plant, botanically known as K. blossfeldiana×K. laciniata interspecific hybrid, hereinafter referred to by the cultivar name ‘KJ 2003 0638’. As used herein, “interspecific hybrid” includes the progeny from the cross of two different species of Kalanchoe, as well as, the progeny resulting from subsequent backcrossing to one of the parents.

The genus of Kalanchoe belongs to the sedum family (Crassulaceae). There are more than 100 different species of Kalanchoe, of which more than 60 are found growing wild on Madagascar, many in South Africa and a few in Asia and South America. Kalanchoe belongs to the succulent plants, which are characterized by having turgid leaves. The leaves enable them to stand drought in nature or on the windowsill for a longer time than most other plants, and this allows for a supreme longevity.

The new Kalanchoe cultivar is a product of a controlled breeding program conducted by the Inventors, Knud Jepsen and Ellen Christensen, in Hinnerup, Denmark. The objective of the breeding program was to create new Kalanchoe cultivars with large flowers, numerous petals per flower, attractive flower coloration and excellent postproduction longevity.

The new Kalanchoe cultivar originated from a cross made in a controlled breeding program by the Inventors in Hinnerup, Denmark. The female parent is a proprietary selection of K. blossfeldiana×K. laciniata interspecific hybrid designated ‘KJ 2000-0716’ having single-type flowers with 4 petals per flower. The female parent is described in pending U.S. Plant patent application Ser. No. 10/654,571 and in European Union Community Variety Rights application serial No. 2003/0974, and is the second generation progeny of a fertile interspecific hybrid between K. blossfeldiana and K. laciniata. The male parent, designated K. blossfeldiana cultivar ‘Monroe’, is described in U.S. Plant Pat. No. 14,714, and has the multi-petalled double-type flower characteristic. The new Kalanchoe cultivar ‘KJ 2003 0638’ was discovered and selected by the Inventors as a flowering plant within the progeny of the stated cross in a controlled environment in Hinnerup, Denmark.

Asexual reproduction of the new cultivar by vegetative terminal cuttings was first performed in February, 2004, in Hinnerup, Denmark, and has demonstrated that the combination of characteristics as herein disclosed for the new cultivar are firmly fixed and retained through successive generations of asexual reproduction. The new cultivar reproduces true-to-type through asexual reproduction.

### BRIEF SUMMARY OF THE INVENTION

The following traits have been repeatedly observed and are determined to be basic characteristics of the new Kalanchoe cultivar ‘KJ 2003 0638’ which in combination distinguish this Kalanchoe as a new and distinct cultivar:

1. large number of petals per flower resulting in a double-type or multi-petalled trait;

2. large size of petals resulting in a large flower;

3. the hastate, dissected leaves on young as well as mature leaves;

4. the different soft-white and creamy colors of petals and

5. large number of flowers per plant.

The new Kalanchoe cultivar ‘KJ 2003 0638’ has not been observed under all possible environmental conditions. The phenotype of the new cultivar may vary significantly with variations in environment such as temperature, light intensity, and day length without any change in the genotype of the plant. The following observations, measurements and values describe the new Kalanchoe cultivar ‘KJ 2003 10 0638’ as grown in Hinnerup, Denmark, under conditions which closely approximate those generally used in commercial practice.

Plants of ‘KJ 2003 0638’ differ from plants of the parental cultivars in the following characteristics:

| | New Cultivar | Female Parent | Male Parent |
|---|---|---|---|
| Trait | ‘KJ 2003 0638’ | ‘KJ 2000 0716’ | ’Monroe’ |
| Height | About 31 cm | About 38 cm | About 24 cm |
| of cultivar | | | |
| Flower Type | Double-type | Single-type | Double-type |
| Flower | 20-25 mm | 20-25 mm | 16-19 mm |
| Diameter | | | |
| Flower | 250-300 | 500 | 350-400 |
| Number | | | |
| Per Plant | | | |
| Number of | Up to 17 full or | 4 | Up to 27 full or |
| Corolla | partial petals | | partial petals |
| Lobes | | | |
| Corolla | From outer to center | Red-orange, | White, |
| Coloration | of flower: | RHS 44B | RHS 155 D |
| | First circle | | |
| | Yellow-orange, | | |
| | RHS 20D | | |
| | Next circle | | |
| | Yellow-orange, | | |
| | RHS 19B | | |
| | Center circle | | |
| | Yellow-orange, | | |
| | RHS 20D | | |
| | with a twist of red, | | |
| | RHS 39C | | |
| | Whole flower faints | | |
| | to light orange | | |
| | RHS 27C | | |
| Shape of | The width of the | The width of the | The width of |
| petal | petal is 9 mm. | petal is 10 mm. | the petal is |
| | The length is 6 mm. | The length is | 4 mm. The |
| | The shape is ovate | 13 mm. The shape | length is 8 mm. |
| | with mucronate | is obovate with | The shape is |
| | apex. | cuspidate tip. | ovate with |
| | | | cuspidate tip. |
| Leaf | Hastate, dissected | Hastate, dissected | Obovate leaves |
| | leaves with a long | leaves with a long | with obtuse |
| | petiole and long | petiole and long | leaf tips and |
| | lamina. The leaf | lamina. The leaf | truncate bases. |
| | apex is obtuse and | apex is obtuse and | The leaf |
| | the base is cunate. | the base is cunate. | margin is |
| | | | undulate. |
| Leaf | The foliage is | The foliage is | The foliage is |
| texture | glabrous and | glabrous and more | glabrous and |
| | shinning both | shinning above than | shinning both |
| | above and below. | below. | above and |
| | | | below. |

Of the many commercial cultivars known to the present Inventors, the most similar in comparison to ‘KJ 2003 0638’ is parental cultivar ‘KJ 2000 0716’. Comparing these two cultivars, the main distinction is the form and the color of the flower, as described in the preceding table.

### BRIEF DESCRIPTION OF THE DRAWING

The accompanying photographic drawings illustrate the overall appearance of the new Kalanchoe cultivar ‘KJ 2003 0638’ showing the colors as true as is reasonably possible with colored reproductions of this type.

The first photograph shows a top perspective view of a typical potted flowering plant of ‘KJ 2003 0638’, 18 weeks after planting of cutting. The second photograph shows a side perspective view of a typical potted flowering plant of ‘KJ 2003 0638’, 18 weeks after planting of cutting. The third photograph shows the following typical plant parts of ‘KJ 2003 0638’: A Inflorescence; B. Flower, just opened; C. Flower opened one week, D. Flower bud, top; E. Inside a flower; F. Flower bud, site; G. Petal; H. Pistil; I. Sepal; J. Mature leaf; and K. Young leaf.

### DETAILED BOTANICAL DESCRIPTION

‘KJ 2003 0638’ has not been observed under all possible environmental conditions. The phenotype of the new cultivar may vary significantly with variations in environment such as temperature, light intensity, and day length without any change in the genotype of the plant.

The aforementioned photographs, together with the following observations, measurements and values describe the new Kalanchoe cultivar as grown in a greenhouse in Hinnerup, Denmark, under conditions which closely approximate those generally used in commercial practice. The plants were grown in a greenhouse at 64.4° F. at daytime and 68° F. during the night. The cultivar was produced in a either a 10.5 or 13 cm pot. The cuttings were grown in a long day (16 hours light, 8 hours dark) for the first 5 weeks. At week 6 after planting, the cultivar is grown in a short day (10 hours light, 14 hours dark). After 14 weeks the cultivar would be ready for sale. The cultivar was grown under natural light conditions supplemented with 70 μmol/m²/s SON-T light when the natural light was less than 100 μmol/m²/s. At short day, the flowering was induced. The reaction time from day of induction to day of first opened flower was 67 days. The peat based soil mix was watered with a solution containing 200 parts per million (ppm) nitrogen, 200 ppm potassium and 40 ppm phosphorous, 200 ppm calcium, 40 ppm magnesium, 60 ppm sulphate, 1 ppm iron, 0.6 ppm manganese, 0.1 ppm copper, 0.1 ppm zink, 0.3 ppm borium, 0.03 ppm molybdenum.

In following description, color references are made to The Royal Horticultural Society Colour Chart (RHS), published 1986, except where general colors of ordinary significance are used. Color values were taken under daylight conditions at approximately 12 a.m. in a greenhouse in Hinnerup, Denmark. The age of the plant described is 15 weeks (from the time the cutting was planted in growth medium to when the picture was taken).
Parentage:
Male or pollen parent: K. blossfeldiana cultivar ‘Monroe’ (disclosed in U.S. Plant Pat. No. 14,714)
Female or seed parent: K. blossfeldiana×K. laciniata interspecific hybrid cultivar ‘KJ 2000 0716’ (disclosed in U.S. patent application Ser. No. 10/654,571)
Classification:
Botanical: Kalanchoe interspecific hybrid×K. blossfeldiana cultivar ‘KJ 2003 0638’ (2^{nd} generation after K. blossfeldiana×K. laciniata interspecific hybrid back-crossed with K. blossfeldiana).
Propagation: Vegetative terminal cuttings.
   Rooting Habit.—The cultivar has numerous, branched, fine and fibrous roots.
   Time to initiate roots.—It takes between one and two weeks to initiate roots.
   Time to produce a rooted cutting.—It will take three weeks to produce a well rooted cutting. In winter time, it can take one week more.
Plant: If the plants are grown according to the description above, it will perform as described below. Variation from this should be expected over the course of the year, and if the physical growing conditions varies from the description.
   Growth habit.—This cultivar is upright and uniform. The flowers are formed above the top of the leaf canopy, but flowers will also appear between the leaves. The flowers are assembled in numerous compound inflorescences.
   Growth rate.—Ready for sale after 16 weeks.
   Branching habit and description.—Freely branching; typically 4 to 8 lateral branches develop per plant. Pinching (removal of the terminal apex) is not mandatory, but will enhance lateral branching. When grown in 10 cm pots the cultivar is not pinched.
   Height at flowering.—The height of the cultivar is about 31 cm, from the bottom of the pot to the top of the plant, depending on growth conditions.
   Spread/diameter at flowering.—The diameter at flowering is about 30 cm.
Stems:
   Appearance.—The cultivar has around 8 stems with none, one or numerous leaf pairs on the stem. The inflorescence on the top of the flower stem is branched, giving an inverted triangle with a rounded crown. Each branch has between 40 and 60 flowers.
   Aspect.—The stem is strong.
   Length.—The length of the stem is about 14 cm depending on growth conditions.
   Texture.—The texture of the stem is glabrous and shinning.
   Color.—The color of the stem is yellow-green, RHS 146 A.
Foliage:
   Arrangement.—The foliage is yellow-green with the same colors of the immature as well as the mature leaf, RHS 146 A and RHS 147 A. The majority of the leaves are at the base of the plant, but immature leaves appear also on the flowering stem.
   Overall shape of leaf.—The leaves are defined as hastate, dissected leaves.
   Apex.—The form of the apex is obtuse.
   Base.—The form of the base is cunate.
   Length.—The length of the foliage varies from about 3 cm for an immature leaf to about 14 cm for a mature leaf.
   Width.—The width varies from about 2 cm for an immature leaf to about 13 cm for a mature leaf.
   Margin.—The foliage has a dentate margin.
   Texture.—The foliage is glabrous and shinning both above and below.
   Color of upper surface.—Mature leaf: The color of the mature leaf is dark yellow-green and is closely described with RHS 147 A. Immature leaf: The immature leaf has the same color as the mature leaf, dark yellow-green, described with RHS 147 A.
   Color of lower surface.—Mature leaf: The color of the lower surface is different from the upper surface, and is closely described with a dark yellow-green color between RHS 146 A and RHS 147 A. Immature leaf: The immature leaf has the same color as the mature leaf, dark yellow-green, and is closely described with a dark yellow-green color between RHS 146 A and RHS 147 A.
   Venation color.—There is no visual appearance of veins. Upper surface: There is no difference between the color of venation and the rest of the leaf. Lower surface: There is no difference between the color of venation and the rest of the leaf.
   Petiole.—Length: The length of the petiole varies between an immature and mature leaf. The length of an immature leaf varies between 0.5 and 1 cm. The length of a mature leaf varies between 3 and 3.5 cm. Diameter: The diameter of petiole varies between an immature and mature leaf.

The diameter of an immature leaf is 4 mm. The diameter of a mature leaf is 5 mm. Color: There is no difference between the color of petiole and the rest of the leaf.
Fower description:
   Flower type and habit.—The flower type is a double-type with 16 to 19 petals per flower. The average number of petals per flower is 17.
   Natural flowering season.—The flowering season is year round, when grown according to description above. When planting outside the flowering season will be in the summer (April to October), depending on local climatic conditions.
   Time to flower.—The time from start of short day treatment to 1^{st} flower is about 74 days, approximately 1 week longer in winter time.
   Flowering stem length.—The length of the flowering stem is ranging from 13 to 14 cm depending on the growth conditions.
   Post production longevity.—‘KJ 2003 0638’ maintain good leaf and flower substance for at least 6 weeks under interior environmental conditions.
   Winter hardiness/weather tolerance.—‘KJ 2003 0638’ withstands wind, rain and direct sunlight, and can cope with temperatures between 40 and 95 degrees F.
   Fragrance.—‘KJ 2003 0638’ has no fragrance.
   Flower size.—The diameter of the flower range from is 2.5 to 3 cm .
   Overall shape.—The shape is rose-like, defined by at least 5 petals per flower.
   Quantity.—‘KJ 2003 0638’ produces a large number of flower ranging from 200 to 250 flowers per plant.
   Bud.—Rate of opening: From the time when the bud is showing color, the flower will open within 10 days. Color: At the tip the bud is yellow-green, RHS 145 B, with a twist of orange 24 A. At the base the bud has the same yellow-green color, RHS 145 B. Shape: The shape of the bud is ellipsoidal with the smallest diameter towards the base. Length: The length of the bud is about 1.5 cm. Diameter: The diameter of the bud is about 8 mm at the tip and about 4 mm at the base.
   Petal.—Quantity: Typically 16-17 fused at the base. Shape: The shape of petal is oval. Length: The length is about 9 mm. Width: The width is about 6 mm. Apex: The shape of apex is mucronate. Margin: The shape of the margin can be described as entire. Texture: The texture of the petals is soft, smooth and mat. Color when opening: Upper surface: The color of the upper surface of the petals can be described as follows: Going from the outer circle to the inner circle of petals, the first circle of petals is yellow-orange, RHS 20 D, the next is yellow-orange, RHS 19 B, and the middle is yellow-orange, RHS 20 B, with a touch of red, RHS 39 C. Lower surface: The color of the lower surface of the petals is yellow-orange, RHS 23 D, with a touch of red, RHS 38 A.
   Color when fully open.—Upper surface: At maturity the upper surface of the whole flower fades to orange RHS 27 C. Lower surface: At maturity the lower surface of the whole flower fades to orange RHS 27 C.
Sepal:
   Quantity.—Typically 4 sepals fused at the base.
   Shape.—The shape of sepal is lanceolate.
   Length.—The length is 10 mm.
   Width.—The width is 3 mm.
   Apex.—The shape of the apex is acute.
   Margin.—The shape of the margin is entire.
   Texture.—The texture of the sepals is glabrous and shinning.
   Color when opening.—Upper surface: The upper surface of the sepal is green, RHS 137 B. Lower surface: The lower surface of the sepal is green, RHS 137 B.
   Color when fully open.—Upper surface: The upper surface of the sepal is green, RHS 137 B. Lower surface: The lower surface the sepal is green, RHS 137B.
Peduncle:
   Length.—Every flower has a peduncle with a length of 5 mm.
   Color.—The peduncle is yellow-green; RHS 146 A.
   Texture.—The peduncle is glabrous and shinning.
Reproductive organs:
   Stamen.—Number: The flower has between 7 to 9 stamens. Color: The color of the stamens is greyed-orange, RHS 177 B.
   Anthers.—Number: The number of anthers is 7 to 9. Size: The anthers are less than 1 mm in length. Color: The color of the anthers is yellow-brownish, RHS 14 B.
   Filament color.—The color of the filament is yellow-green, RHS 145 C.
   Pollen color.—The color of the pollen is yellow, RHS 17 C.
   Pollen amount.—A medium amount of pollen with very good pollen fertility of 100 % depending on time of year.
   Pistil.—Number: The number of pistils is four.
   Stigma.—Shape: The shape of the stigma is round. Color: The color of the stigma is yellow-green, RHS 145 A.
   Style.—Shape: The shape of the style is thin and cylindrical. Color: The color of the style is yellow-green, RHS 145 A.
   Ovary.—Color: The color of ovary is green, RHS 144 B.
   Seeds.—Number: Between 50 and 60 potential seeds per ovary. Width: The width of the seeds is less than 0.5 mm. Length: The length of the seeds is less than 1 mm. Shape: The shape of the seeds is ellipsoidal. Color: The color of the potential seeds is green, RHS 145 C.
   Fruit (ovary).—Shape: The shape of the fruit is cylindrical. Width: The width of the fruit is 1 mm. Length: The length of the fruit is 6 mm. Color: The color of the fruit is yellow green, RHS 145 A.
Disease Resistance/susceptibilities: No information on disease resistance or susceptibility is currently available.

I Claim:

1. A new and distinct K. blossfeldiana×K. laciniata interspecific hybrid plant named ‘KJ 2003 0638’, substantially as illustrated and described herein.
